(19) United States
(12) Patent Application Publication
(10) Pub. No.: US 2004/0014028 A1
(43) Pub. Date: Jan. 22, 2004

(54) Method for determination of protective epitopes for vaccination, diagnosis and vaccine quality control

(75) Inventors: Osvaldo Jorge Lopez, Lincoln, NE (US); Matias Ostrowski, Buenos Aires (AR)

Correspondence Address: VINCENT L. CARNEY LAW OFFICE, P.O. BOX 80836, LINCOLN, NE, 68501-0836, US

(73) Assignee: Wolf Biotech, Lincoln, NE

(21) Appl. No.: 10/199,545

(22) Filed: Jul. 19, 2002

(51) Int. Cl. C12Q 1/70; G01N 33/53; G01N 33/554; G01N 33/569

(52) U.S. Cl. 435/5; 435/7.32

(57) ABSTRACT

To provide a quality control test for quality of vaccines against a pathogen, a test to determine the presence of antibodies against the pathogen to determine the status of protection of individuals in a herd or a vaccine against the pathogen, in one technique for pathogens having a delayed introduction of protective antibodies, sera from an infected animal is obtained before the eliciting of neutralizing antibodies and after the eliciting of neutralizing antibodies. The two sera are used to remove the non-neutralizing epitopes from the mixture of neutralizing epitopes and non-neutralizing epitopes to obtain pure neutralizing epitopes such as for example by affinity chromatography. In another technique for those pathogens not having the delayed production of protective antibodies, sera is obtained from a protein eliciting neutralizing antibodies and non-neutralizing antibodies to a particular pathogen and serum is obtained from a similar protein that will not elicit neutralizing antibodies against that particular pathogen. The antibodies present in the serum that does not elicit neutralizing antibodies against that particular pathogen are used to eliminate the non-neutralizing eptopes.

### STATEMENT REGARDING FEDERALLY FUNDED RESEARCH

This invention was made, in part, with government support under grant number USDA CSREES/SBIR 2001-33610-10337 awarded by the United States Department of Agriculture. The United States government has certain rights in this invention.

### BACKGROUND OF THE INVENTION

This invention relates to vaccines, methods of diagnosis and methods of vaccination.

The immune system has evolved to respond to the constant challenge that vertebrates suffer from more simple forms of life. A successful immune system will be the one allowing the species to survive these challenges before the reproduction period. For example, in the case of virus, an effective immune response to a viral infection is a new steady-state relationship between the virus and the host that does not compromise the life of the animal (Bachmann M F & Zinkernagel, R. Ann. Rev Immunol 15: 235-70,1997).

During infection, antibodies against different fragments of the different molecules of the pathogen (epitopes) are made. Some of these antibodies are protective but others do not protect against infection, and in some cases, antibodies could be harmful to the host. Once the animal has subdued the viral infection, neutralizing antibodies will hamper a new infection with this virus. Then, the existence of neutralizing (protective) antibodies will prevent infection in most cases. In fact, the majority of effective vaccines against viruses protect via preexisting neutralizing antibodies (Bachmann M F & Zinkernagel, R Ann. Rev. Immunol. 15: 235-70,1997).

Neutralizing antibodies are important to abort infection also of viruses that infect cells belonging to the immune system such as macrophages but specific non-neutralizing antibodies could be harmful to the host. For example, horses infected with equine infectious anemia virus, have episodes of viremia when new, neutralizing-antibody-escape mutants re-infect macrophages. This viremia is cleared when the animal produces neutralizing antibodies towards the new mutant (Rwambo P M, et al, Arch Virol 111: 199-212, 1990). In another example, pregnant macaques treated with a mix of three neutralizing monoclonal antibodies to HIV were protected upon challenge with the virus (Baba T W, et al, Nat Med. 6: 200-6, 2000). Moreover, their infants, who received the cocktail after birth, were also protected against challenge with the virus. The presence of neutralizing, agglutinating, complement fixating, blocking receptor or the like antibodies also prevent infection with other pathogens such as bacteria, parasites and the like.

Vaccines are tools which induce an immune response against a certain pathogen so that a new state of immunity is achieved by the host. A good vaccine should attempt to avoid infection before the virus, bacteria or other pathogen infects the animal. Attenuated vaccines, which are obtained by biological means or by DNA recombinant techniques from wild type viruses, have several advantages over inactivated vaccines. They are cheaper to produce and one injection is normally enough to confer protection against many viruses. It is commonly believed that since these vaccines resemble a natural infection, they should be better in conferring protection. This is true with some viruses, mostly those that induce a high level of neutralizing antibodies soon after infection such as Transmissible Gastroenteritis Virus (TGE), Poliovirus etc. However, other viruses such as PRRSV or Human Immunodefficiency Virus (HIV), have developed a strategy of delayed induction of neutralizing antibodies during the natural infection. Thus, vaccination with an attenuated virus resembling infection will not necessarily be effective in inducing neutralizing antibodies to protect from infection. In addition, attenuated vaccines can revert to the virulent pathogen in the field.

The induction of an immune response similar to a natural infection is not necessarily effective against other pathogens. For example, the variability of the immunodominant antigens against Neumoccocus had hampered the development of vaccines against this bacterium for many years. Using genomics and proteomics, more than 300 antigens expressed from a cDNA library of Neumococcus, were used to produce antibodies in mice. Although complement-fixing antibodies to those previously known immunodominant antigens were elicited, antibodies to other new antigens that also fix complement were elicited too (Pizza, M et al, Science, 287: 1816-20, 2000). Antibodies against these antigens are not present in high levels in a normal infection, and, contrary to the known immunodominant antigens, they are well conserved. Thus, reproducing the natural immune response to a bacterial infection is not necessarily the most expedient way to prevent infection. If these antibodies against these antigens prove to be protective, a new generation of neumococci vaccines could arise from this discovery. This is also the case for some parasites. For example, in Trypanosoma cruzi, the immunodominant epitopes in the trans-sialidase protein (which plays an essential role in infection) are not important in the catalytic activity of this enzyme. The catalytic domain is not immunogenic in a normal infection. However, the induction of antibodies against it protect from infection. (Alvarez P, et al. Infect Immun. 69(12):7946-9, 2001). Thus, a vaccine inducing antibodies to this fragment of this protein could prevent or attenuate infection with this protozoa.

Currently procedures to determine which fragments of a molecule (epitopes) induce antibodies are known in the art. However, these methods have a disadvantage in that they fail to distinguish epitopes that produce neutralizing antibodies from those that produce non-neutralizing antibodies.

Vaccines have been developed against PRRSV. This virus is the etiological agent for the Porcine Reproductive and Respiratory Syndrome of swine. This disease was recognized in the USA in 1987 as a new disease of swine causing late-term reproductive failure and severe pneumonia in neonatal pigs (Keffaber K K. 1989. AASP Newsletter 2: 1-10). The National Pork Producers Council has recognized the Porcine Reproductive and Respiratory Syndrome (PRRS) as being the most important infectious disease affecting swine in North America in recent times (National Pork Producers. (2000) Porcine Reproductive and Respiratory Syndrome Virus NPPC Position Statement, Research and Production Issues. 2000. Published on line by National Pork Producers Council (NPPC), Washington D.C.,(http://www.porkscience.org/documents/other/positionprrs.pdf). In the last fifteen years it has spread around the world causing serious damage to the swine industry.

The commercial PRRSV vaccines currently in use consist of conventional live attenuated strains. They confer protection against clinical disease with highly virulent isolates but not against infection (Osorio, F. A., Allen D. Lennan Swine conference Vol 25: 179-182, 1998). In addition, analysis of field isolates obtained after the introduction of the attenuated vaccine (VR2332) demonstrated that the majority of the isolates were descendants of the vaccine (Oleksiewicz, M. B., et al.. Virology 15:135-140,2000; Wu, W-H et al. Virology, 287(1):183-191, 2001). Thus, it is necessary to fully evaluate the safety and efficacy of the current modified live vaccines. There is a general consensus that there is a need to produce new generation vaccines against PRRSV ((Meng, X. J. Vet. Microbiol. 74:309-329,2000. Mengeling W L, et al.Am J Vet Res. 60: 796-801,1999). These vaccines should: (1) be antigenically broad enough to induce an effective immune response to the vast majority of isolates of PRRSV; (2) induce a vigorous neutralizing antibody response in order to protect animals from infection; and (3) avoid the virulent vaccine revertants that have been described.

It has been found that the onset of neutralizing antibodies after experimental infection is accompanied by clearance of the virus and cells producing gamma-interferon (Labarque, G. G., et al. J. Gen. Virol. 81:1327-1334, 2000). In addition, vaccination of pigs with a DNA vaccine encoding PRRSV GP5 induced neutralizing antibodies although in low levels. Nevertheless, the pigs were protected against challenge with the homologous virulent strain. They presented a mild fever and virus could be recovered after second passage in MARC-145 cells only from lungs and mediastinal lymph nodes.

These results demonstrate that neutralizing antibodies are important in protection against the disease. In fact, the neutralizing antibody response after vaccination with the DNA vaccine resembles the response to an attenuated strain of PRRSV: after vaccination with a modified live vaccine there is a low or non-detectable neutralizing antibody responses which rapidly raises in titer upon challenge with virulent PRRSV.

Moreover, the results of a passive transfer of antibodies performed recently in pregnant gilts (Osorio, F. A., et al. Virology, in press) unambiguously indicate that the sole transfer of a solution enriched in neutralizing antibodies conferred complete protection against reproductive failure induced by PRRSV. These results clearly demonstrate that the antibodies prevented the infection of the gilts and their offspring. These animals were protected not only against disease but also against infection, providing sterilizing immunity (prevention of infection of the females and no transmission of PRRSV to the offspring). Thus, a successful vaccine against PRRSV will be one that will induce a high level of neutralizing antibodies against GP5.

An epitope that produces neutralizing antibodies (neutralizing epitope} has been found in protein GP5 of PRRSV. In addition, an immunodominant epitope has been found that is not neutralizing and works as a decoy epitope. Others viruses such as HIV (Garrity, R. R., 1997 J. Immunol. 159:279-289) have developed a similar strategy to escape from a protective immune response: close to a neutralizing epitope they carry an immunodominant, highly variable and non-neutralizing decoy epitope. The presence of this decoy epitope distracts the protective (neutralizing antibodies) immune response of the host early in the infection. It is believed that these viruses use this strategy to avoid neutralization of epitopes that have an important function (i.e. interaction with the cellular receptors and penetration into the host cell) for the virus and for which the virus cannot afford antigenic drift to avoid the immune system's surveillance. Thus, these Inventors have discovered that immunization with attenuated vaccines or vaccines including proteins or glycoproteins or their genes can elicit an immune response against certain epitopes present in PRRSV's vaccines that could hamper the induction of an effective immune response. This finding explains the lack of success with the commercially available vaccine.

At present the farming industry is adversely affected by the economic damage caused by PRRSV infection. There is a trend away from the use of vaccines. Instead, more expensive management procedures are being employed to avoid economic losses caused by Porcine Reproductive and Respiratory Syndrome. Thus, it is desirable to find: (1) a new generation of vaccines in this area for U.S. and worldwide producers, which is an inexpensive way to fight to this disease; (2) tests that will allow the determination of the status of protection of individuals in a herd and to determine the quality of the immunogen present in a vaccine; (3) tests to determine if those individuals in a herd with anti-PRRSV antibodies are vaccinated or infected with the virus; (4) monospecific antibodies that will neutralize the virus for use in prevention; and (5) to obtain the public health benefit of using a vaccine as a way to avoid infection with PRRSV of livestock used for human consumption.

### SUMMARY OF THE INVENTION

Accordingly, it is an object of the invention to provide a general method for discriminating between epitopes that induce neutralizing antibodies from those that induce non-neutralizing antibodies.

It is a further object of this invention to provide a general method to determine protective epitopes in molecules present in pathogens.

It is a still further object of the invention to provide a novel vaccine.

It is s still further object of the invention to provide a novel method of designing new vaccines.

It is a still further object of the invention to provide a novel method of quality control of vaccines.

It is a still further object of the invention to provide a novel method for determining the status of protection of a population by determining the amount of antibodies against this particular epitopes carried by the population.

It is a still further object of the invention to use peptides mimicking (mimotopes) neutralizing epitopes in a vaccine.

It is a further object of the invention to provide a novel method for determining what areas of a pathogen should be added and what fragment should not be added to a vaccine in order to make it effective in the induction of protection against the infection with the pathogen.

It is still a further object of the invention to provide a novel method for vaccination.

It is a still further object of the invention to provide a method for vaccination where a vaccine eliciting neutralizing antibodies against a certain neutralizing epitope of a pathogen is used in conjunction with neutralizing antibodies against the pathogen directed against different neutralizing epitopes.

It is still a further object of the invention to provide a differential kit for diagnosis of antibodies that will allow the determination of whether the anti-pathogen antibodies present in the fluids of an animal confer protection or do not confer protection in the case of infection with the pathogen.

It is still a further objective of the invention to provide a differential kit for diagnosis which will allow a diagnosis of whether an animal has anti-pathogen antibodies as a consequence of an infection or as a consequence of vaccination against the pathogen.

It is still a further object of the invention to provide a tool to determine if a vaccine against a pathogen has the epitopes/mimotopes needed to elicit neutralizing antibodies and therefore, will induce a protection state in vaccinated animals.

It is a still further object of the invention to provide a general method for discriminating between epitopes that induce neutralizing antibodies to PRRSV from those epitopes that induce non-neutralizing epitopes from PRRSV.

It is a further object of this invention to provide a general method to determine protective epitopes in molecules present in PRRSV.

It is a still further object of the invention to provide a novel vaccine against PRRSV.

It is a still further object of the invention to use peptides mimicking (mimotopes) neutralizing epitopes in a vaccine against PRRSV.

It is a further object of the invention to provide a novel method for determining what areas of glycoprotein GP5 of PRRSV should be added and what fragment should not be added to a vaccine in order to make it effective in protection against the infection with PRRSV.

It is a still further object of the invention to provide a method for vaccination where a vaccine eliciting neutralizing antibodies against a certain neutralizing epitope/mimotope of PRRSV is used in conjunction with neutralizing antibodies against PRRSV directed against different neutralizing epitopes.

It is still a further objective of the invention to provide a differential kit for diagnosis of PRRSV antibodies that will allow the determination of whether the anti-PRRSV antibodies present in the fluids of an animal confer protection or do not confer protection in the case of infection with PRRSV.

It is still a further objective of the invention to provide a differential kit for diagnosis which will allow a diagnosis of whether an animal has anti-PRRSV antibodies as a consequence of an infection or as a consequence of vaccination.

It is still a further objective of the invention to provide a tool to determine if a vaccine against PRRSV has the epitopes needed to elicit neutralizing antibodies and therefore, will induce a protection state in vaccinated animals.

It is a still further object of the invention to provide a novel vaccine to protect against infection with PRRSV.

It is a still further object of the invention to provide the sequence of a protein fragment from GP5 of PRRSV that should be included and protein fragments that should not be included in a vaccine to protect against infection with PRRSV.

It is a further object of the invention to provide a novel method to detect those epitopes which elicit non-neutralizing antibodies and those epitopes which elicit neutralizing antibodies against PRRSV to produce an effective vaccine against PRRSV.

It is a still further object of the invention to provide a technique for isolating neutralizing epitopes of a pathogen and obtain such epitopes substantially free of non-equalizing epitopes.

In accordance with the above and further objects of the invention, a vaccine includes a neutralizing epitope and does not include non-neutralizing epitopes to a pathogen. The vaccine is obtained from either of two methods. Firstly, for those pathogens, such as PRRSV, that have a delayed production of neutralizing antibodies, sera from an infected animal is obtained before the eliciting of neutralizing antibodies and after the eliciting of neutralizing antibodies. The two are used to remove the non-neutralizing epitopes from the mixture of neutralizing epitopes and non-neutralizing epitopes to obtain pure neutralizing epitopes such as for example by affinity chromatography. Secondly, for those pathogens not having the delayed production of neutralizing antibodies, sera is obtained from a protein eliciting neutralizing antibodies and non-neutralizing antibodies to a particular pathogen and serum is obtained from a similar protein that will not elicit neutralizing antibodies against that particular pathogen. The antibodies present in the serum that does not elicite neutralizing antibodies against that particular pathogen are used to eliminate the non-neutralizing eptopes.

By these general methods, several isolations and determinations are made, such as: (1) the isolation and characterization of a neutralizing epitope for a pathogen which should be included in a vaccine against the pathogen; (2) the isolation and characterization of an epitope that distracts the antibody response against the pathogen and should not be included in this vaccine; and the characterization of the use of the neutralizing epitope/mimotope for quality control of vaccines against the pathogen, for a diagnostic test to be used to assess the status of protection against infection by the pathogen on individuals in a herd and as a target for passive transfer of antibodies to cure, decrease shedding or prevent infection with the pathogen.

In one embodiment, a fragment of GP5 of PRRSV that should be included in a vaccine that will protect swine from the infection with PRRSV is obtained and a fragment of PRRSV GP5 that must not be in the vaccine because distracts the antibody response from the protective epitope/mimotope is obtained. A quality control test is provided that will allow the determination of the presence of the fragment of GP5 that induces neutralizing antibodies against PRRSV to be used to check for quality of vaccines. A test is provided to determine the presence of antibodies against the fragment that elicits neutralizing antibodies to be used as a way to determine the status of protection of individuals in a herd. Monospecific antibodies against the fragment of GP5 of PRRSV inducing neutralizing antibodies may be used to cure, decrease viral shedding or prevent infection with PRRSV of an adult pig or its embryos. However, the method used to determine these protective epitopes/mimotopes could be successfully applied to determine protective epitopes/mimotopes to other pathogens in swine and other species.

This method resembles the natural immune response to the wild-type virus and confers protection to a second infection by the same pathogen or protects the offsprings by the mother's milk or colostrum. Thus, a vaccine inducing neutralizing antibodies will be effective in preventing infection against most viruses.

From the above description it can be understood that the vaccines and methods of this invention have several advantages, such as for example: (1) the vaccines provide better protection; (2) it is useful in designing new vaccines; (3) it is useful for quality control of vaccines; (4) it is useful in the production of marker vaccines allowing differentiation of vaccinated from infected individuals; and (5) it is useful in determining the status of protection of a population by determining the amount of antibodies against this particular epitopes carried by the population.

### BRIEF DESCRIPTION OF THE FIGURES

The above noted and other features of the invention will be better understood from the following detained description when considered with reference to the accompanying drawings, in which:

FIG. 1 is a diagrammatic view of a process of screening for a protective epitope in accordance with an embodiment of the invention;

FIG. 2 is a diagrammatic view of another process of screening for a protective epitope in accordance with another embodiment of the invention;

FIG. 3 is a chart showing the deduced amino acid sequences of relevant peptides carried on phage clones obtained in four different selections;

FIG. 4 is a chart illustrating the reactivity of swine sera and monoclonal antibodies to selected cloned phages;

FIG. 5 is a graph with a curve showing the inhibition by synthetic peptides of the binding of monoclonal antibody ISU25C1 (solid lines) a curve showing monoclonal antibody ISU19-A1 (dotted lines) to PRRSV in ELISA in cartesian coordinates with ordinates of percentage of specific signal and abscissa of jpeptide concentartion (micromolar);

FIG. 6 is a chart illustrating the reactivity of Monoclonal antibody ISU25-C1 and swine anti-PRRSV hyperimmune sera to synthetic peptides bound to MBS coated ELISA plates;

FIG. 7 is a chart illustrating the reactivity of anti peptides-affinity purified antibodies to synthetic peptides bound to MBS coated ELISA plates.

FIG. 8 is a graph having curves in Cartesian coordinates with ordinates of percentage of specific signal and abscissa of peptide concentration in micromoles showing the inhibition by synthetic peptides of the binding of anti-peptide S2-affinity purified antibodies to phage clone S2-4.

FIG. 9 is a chart illustrating the specific recognition, by two sequential sera samples a from six hyperimmunized pigs, of epitopes A and B.

FIG. 10 diagram comparing the predicted amino acid sequences of GP5 of PRRSV and LDV.

FIG. 11 is a Western Blot of purified PRRSV proteins digested with endoglycosidase-F; and

FIG. 12 are three charts with ordinates of amount of binding and abscissa of concentration of S2 or R1N and three different titer.

### DETAILED DESCRIPTION

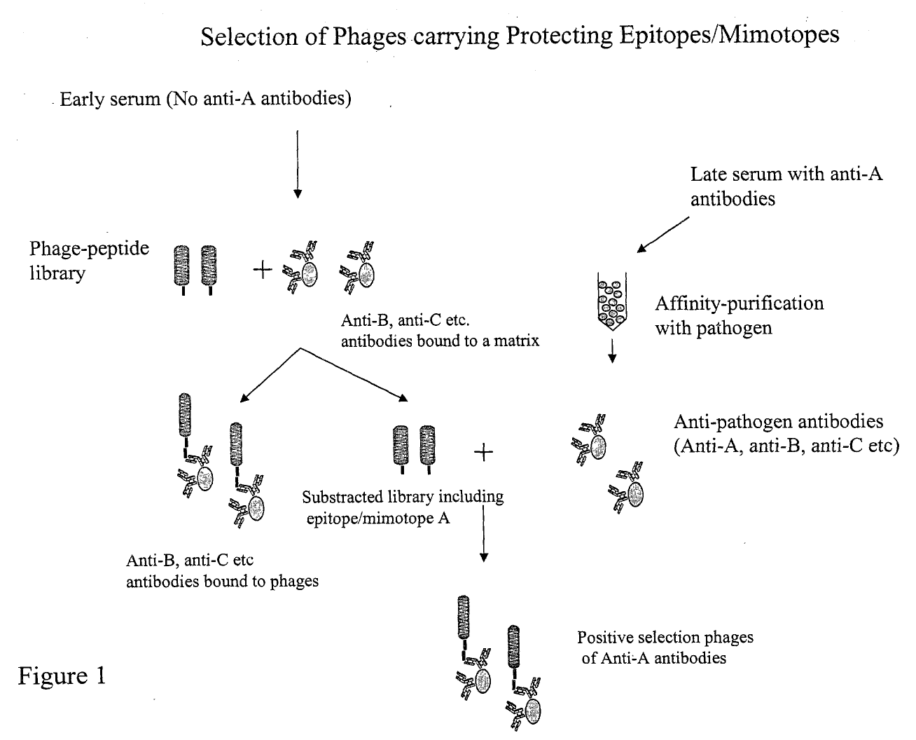

In FIG. 1, there is shown an example of screening for a protective epitope taking advantage of the delay of the natural antibody response to a infection by a certain pathogen. Epitope A is a protective epitope/mimotope while epitopes B and C are non-protective epitopes/mimotopes located in the same pathogen. A serum with noneutralizing activity is used to deplete a phage display library of peptides to those epitopes/mimotopes such as epitopes B and C. Then, a serum with neutralizing activity is first affinity-purified against the pathogen and these antibodies are used to screen the library subtracted of non-neutralizing peptides/mimotopes. In the particular case of a phage display library shown in this example, the DNA fragment encoding for the particular peptide on each individual phage clone is sequenced and the deduced amino-acid composition is compared to proteins of the pathogen.

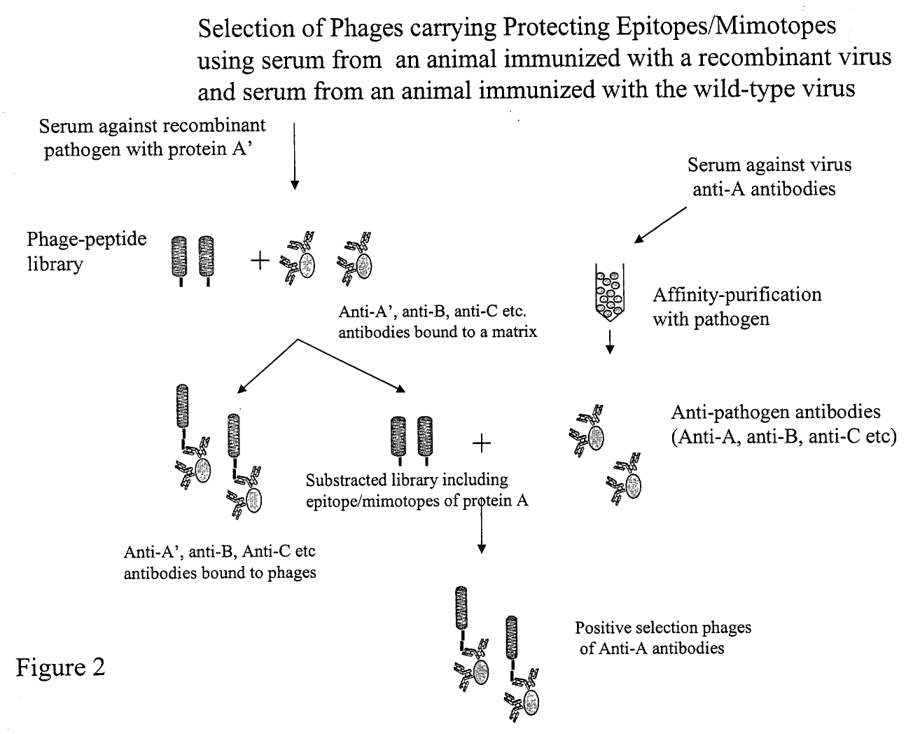

In FIG. 2 there is shown a diagrammatic view of another process of screening for a protective epitope by using a virus where a protein (A) inducing protective antibodies against the target virus is changed for a similar protein (A′) from another virus or viral isolate. Protein A′ will not induce neutralizing epitopes to the target virus. In this example, this new virus containing all the proteins except protein A is obtained by genetic manipulation. Serum from an animal immunized with the recombinant virus will produce antibodies against epitope A′ and against non-protective epitopes B and C. This serum is used to deplete a phage display library of peptides to those epitopes/mimotopes such as epitopes B and C. Then, serum from an animal immunized with the target virus and, thus, having anti-A antibodies is first affinity-purified against the virus and these antibodies are used to screen the library subtracted of non-neutralizing peptides/mimotopes.

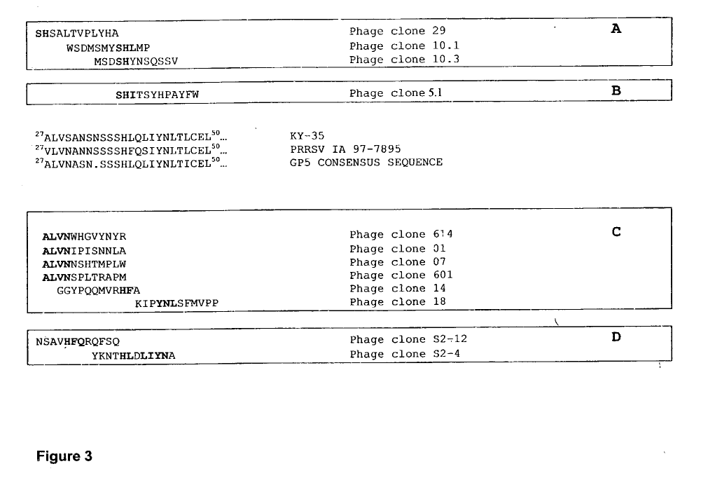

In FIG. 3 there is shown an deduced amino acid sequences of relevant peptides carried on phage clones obtained in four different selections. Peptides expressed by phages selected with monoclonal antibody ISU25C-1 under low (A) or high (B) stringency conditions, with swine antibodies possessing high SN titer (C) or with swine anti-peptide S2-affinity purified antibodies (D) are shown aligned with PRRSV Iowa strain, PRRSV KY-35 and a consensus sequence deduced from the ORF 5 products of several North American strains (Pizardeh, D and Dea, S, Can. J. Vet. Res. 62: 170-177, 1998). The sequences are given using the standard single letter aminoacid code.

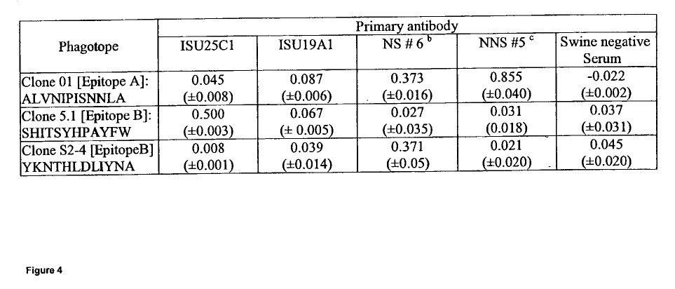

In FIG. 4 there is shown the reactivity of swine sera and monoclonal antibodies to selected cloned phages. Results are expressed as corrected OD₄₀₅ (OD of selected phage-OD unselected phage library NS: serum from pig #6 with a neutralizing titer >64 and ELISA s/p ratio 1.053 NNS: serum from pig #5 with a neutralizing titer <4 and ELISA s/p ratio 0.652.

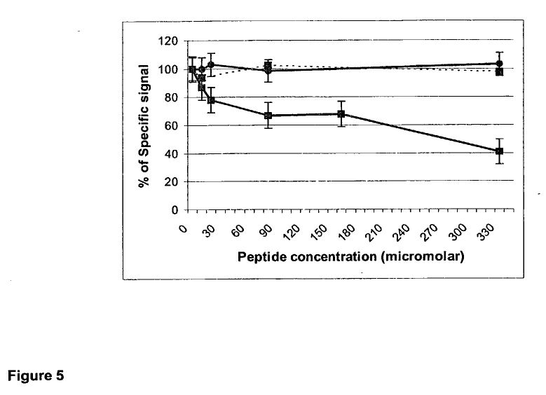

In FIG. 5 there is shown the inhibition by synthetic peptides of the binding of monoclonal antibody ISU25C1 (solid lines) or monoclonal antibody ISU19-A1 (dofted lines) to PRRSV in ELISA. Serial five-fold dilutions of synthetic peptide S4 were used (filled squares). Unrelated peptide P7 was used with monoclonal antibody ISU25C1 as a negative control (filled circles). Results are expressed as the percentage of the anti PRRSV-specific signal obtained with each monoclonal antibody in wells to which no peptides were added. Values are the average of four readings. The sequences of peptides S4 and P7 are shown in Table 1.

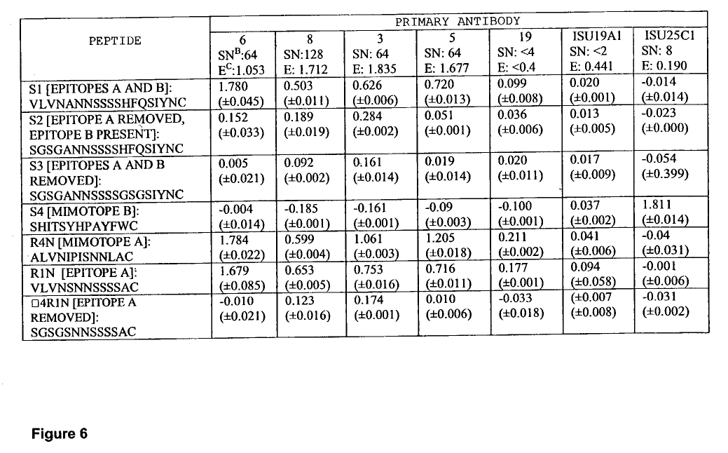

In FIG. 6 there is show the reactivity of Monoclonal antibody ISU25-C1 and swine anti-PRRSV hyperimmune sera to synthetic peptides bound to MBS coated ELISA plates. Results are expressed as corrected Optical density (OD) at 405 nm (OD405): (OD tested peptide-OD irrelevant peptide P7, Table 1). SN titer was determined against PRRSV Iowa strain. ELISA s/p ratio was determined as the difference in the optical density (OD) of the test serum between viral and control antigens divided by the difference in the OD of the positive reference between viral and control antigens. Serums 3, 5, 6 y 8 are from PRRSV-hyperimmunized pigs, serum 19 is a negative control, ISU 25-C1 and ISU 19A1 (Yang, L et al, Arch. Virol. 145:1599-1619, 2000) are anti-PRRSV neutralizing and non-neutralizing monoclonal antibodies respectively.

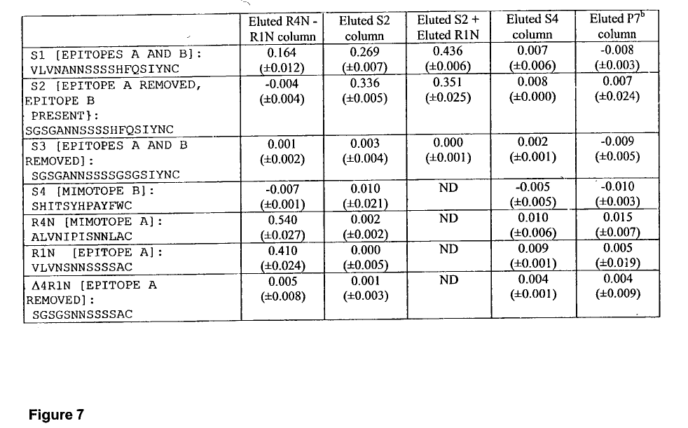

In FIG. 7, there is shown the reactivity of anti peptides-affinity purified antibodies to synthetic peptides bound to MBS coated ELISA plates. Results are expressed as corrected OD405 (OD tested peptide-OD irrelevant peptide P7, Table 1).

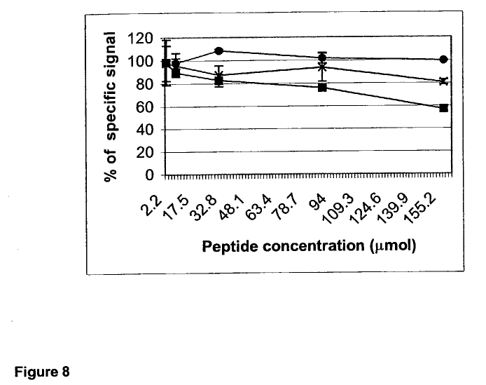

In FIG. 8, there is shown the inhibition by synthetic peptides of the binding of anti-peptide S2-affinity purified antibodies to phage clone S2-4. Serial five-fold dilutions of synthetic peptides S2 (filled squares), S3 (X) or irrelevant peptide P7(filled circle) were used. Results are expressed as the percentage of the signal obtained with anti-peptide S2 affinity purified antibodies in wells to which no peptides were added. Values are the average of four readings. The sequences of peptides S2, S3 and P7 are shown in Table 1. The peptide carry by phage-clone S2-4 is shown in FIG. 3D.

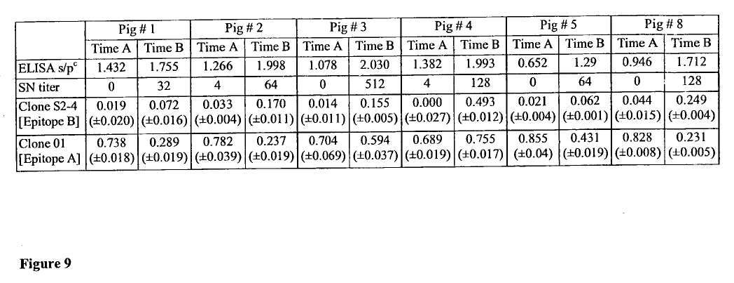

In FIG. 9 shows the specific recognition, by two sequential sera samples a from six hyperimmunized pigs, of epitopes A and B. Time A and Time B correspond to an early (20 days pi) and to an advanced stage (7 months post-immunization) of the hyperimmunization protocol, respectively. Epitopes A and B are represented by phage clones 01 and S2-4 (FIG. 1), respectively. ELISA s/p ratio was determined as the difference in the optical density (OD) of the test serum between viral and control antigens divided by the difference in the OD of the positive reference between viral and control antigens.

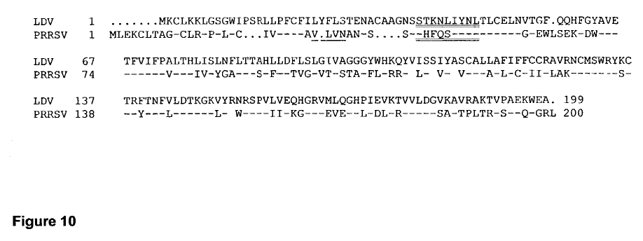

In FIG. 10, there is shown the comparison of the predicted aminoacid sequences of GP5 of PRRSV and LDV. The predicted aminoacid sequences of PRRSV and Lactic Dehydrogenase-elevating virus (LDV). The predicted amino acid sequences of PRRSV isolate Iowa 79-7895 GP5 (Allende R. et al, Arch. Virol. 145: 1149-1161, 2000) and LDV ORF5 (Li, K et al, Virology, 242: 239-245, 1998) are compared. Identical aminoacids are indicated by (−). gaps are indicated by dots. Key residues in epitope A are singled-underlined in PRRSV sequence. epitope area is doubled-underlined both in PRRSV as in LDV. The only residue mutated in GP5 of a neutralization escape mutant of LDV is marked with an arrow.

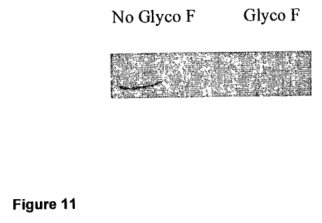

In FIG. 11, there is shown a Western Blot of purified PRRSV proteins digested with endoglycosidase-F which was probed later with monoclonal antibody (Monoclonal antibody) ISU25-C1. Twenty mg of purified PRRSV which was digested with 2 units of Endoglycosidase-F following directions from the manufacturer (Glyco, Novato Calif.). One mg was electrophoresed in a 12% gel of polyacrylamide. Proteins were transferred to a nitrocellulose membrane and probed with Monoclonal antibody ISU25-C1 followed by a peroxydase-conjugated goat anti-mouse-and Lumiglo (KPL,- Gaithersburg) substrate. The reaction was exposed to radiographic film and developed.

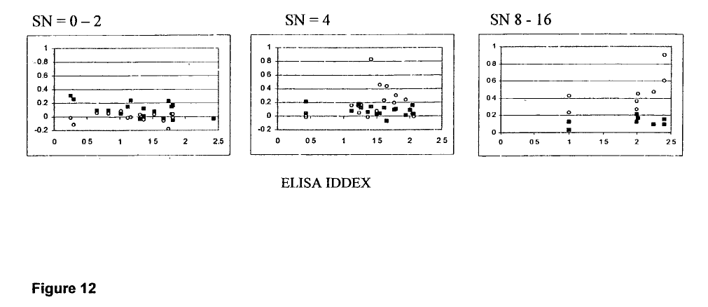

In FIG. 12, there is shown charts that indicate identification of sera from swine having seroneutralization (SN) titer over4 recognize peptide S2. Three charts were made, one representing sera with a SN titer of 0 or 2, a second with a SN titer of 4 and the third representing sera with SN titer of 8 or 16. The data is represented with the OD obtained using a commercial ELISA (IDDEX Lab Inc, Westbrook, Me.) on the X axis and the peptide ELISA S2 (open circle) or R1N (filled square) on the Y axis. The sequences of peptides S2 and R1N are shown in Table 1.

In this specification, an epitope is the area of a molecule, such as a fragment of a protein, of a pathogen which elicits antibodies against it. A mimotope describes those conformational epitopes which resemble the original epitope to which an antibody response was initially mounted (Geysen H M, et al PNAS, 81: 3998-4002, 1984). A neutralizing antibody is that one that interacts with an epitope of a virus inhibiting the infection of susceptible cells by the same virus. A non-neutralizing antibody is an antibody that interacts with an epitope of a virus without inhibition of the infection of susceptible cells by the same virus.

There are different ways known in the art to assess epitopes of a pathogen. One art uses libraries of diverse peptides to screen those peptides binding to antibodies specific for the pathogen. The emergence of solid-phase peptide and display combinatorial libraries allowed for the identification of different epitopes, including carbohydrates (Pinilla, C et al, Current Opinion Immunol., 11: 193-202, 1999).

For example, using solid-phase peptide combinatorial libraries and selecting with a monoclonal antibody against Respiratory Syncytial Virus, several peptides were obtained. Two of them had an affinity constant of more than 109 M-1 with the monoclonal antibody (Chargelegue D, et al, J. Virol., 72: 2040-6, 1998). This approach allows the determination of epitopes that should be in a vaccine or diagnostic test against a pathogen.

To screen for neutralizing epitopes in a combinatorial library, normally, a neutralizing monoclonal antibody made in mouse is used. This approach has three drawbacks. First, it is necessary to have a monoclonal antibody with good neutralizing activity against the virus. Second, mouse antibodies have differences with other species antibodies and the epitopes that they recognize are not necessarily the same as those recognized by antibodies of the species infected by that particular virus. Finally, using a monoclonal antibody to select against a library restricts the search to those epitopes/mimotopes which resembles the epitope to which the monoclonal antibody was produced. The use of polyclonal antisera allows a broadening of the search for epitope/mimotopes which can elicit a good antibody response in the species infected by a particular virus and allows to determine neutralizing epitopes recognized by antibodies of the target species.

There are several methods available to search for libraries of peptides. Progress in chemistry of sugars will permit the search for oligosaccharides in the near future and the search for other molecules and combination of molecules will be available too. In addition, it is also possible to search non-random libraries using fragments of the proteins of the pathogen. These random and non-random display libraries could be expressed on prokaryote or eukaryote systems, allowing, in the lafter, for post-translational modifications of peptides.

During infection or immunization of the species target for a virus, many antibodies with different specificities are elicited. Some are directed against neutralizing epitopes. However, presently there is no art which allows the identification of neutralizing epitopes/mimotopes using antibodies from immune serum of the target species for a particular virus. To overcome this limitation, these Inventors have developed a general method to screen for neutralizing epitopes/mimotopes on virus which could be extended to other pathogens.

The general method consists of: (1) a pool of antibodies from serum against non-protective epitopes of a certain virus are used to subtract non-protective epitopes/mimotopes from a random or a non-random library. There are several ways to obtain antibodies against non-protective epitopes and some formats are described below. Serum obtained by immunization with a virus, will containing protective antibodies will always contain non-protective antibodies too. Then, a serum carrying antibodies against protective epitopes of the pathogen is used to screen the library obtained by subtraction of those antibodies against non-protective epitopes.

The determination of the sequence of these peptides, oligosaccharides etc or mix of different molecules positively selected from the library, allows the determination those epitopes present in this molecule which elicit a protective antibody response against the pathogen.

The invention will be further characterized by the following hypothetical examples. These examples are not meant to limit the scope of the invention which has been fully set forth in the foregoing description.

### Hypothetical Example 1

Some viruses induce an immune response in the natural host with a delay in the production of neutralizing antibodies over the production of non-neutralizing antibodies. Then, serum with non-neutralizing activity is obtained from animals after a few days post-infection. This natural immune response could be used as an advantage to screen for neutralizing epitopes. In the example shown in FIG. 1, antibodies against a neutralizing epitope named A are not present in the serum obtained early after infection. Nevertheless, antibodies against other non-protective epitopes (such as anti-B or anti-C antibodies) are present in this serum. In the example shown in FIG. 1, these early serum antibodies are used to deplete a library of peptides displayed on phages. This library could be a random one (different peptides on each of diverse millions of phages) or a library constructed using fragments of the pathogen. Then, the remaining phages from the library which is now depleted of non-protective epitopes/mimotopes are used further. Concurrently, serum from later in the infection (which contains antibodies against protective epitopes) is positively selected against all epitopes present in the pathogen. This could be done using affinity purification. These antibodies (which are a mix of pathogen-specific protective and non-protective antibodies) are used to screen the library depleted of peptides binding to the early serum antibodies. As a result, only those phages carrying epitopes/mimotopes interacting with the protective antibodies are selected. This procedure exemplified here with a phage library could be performed with different natural or synthetic libraries.

### Hypothetical Example 2

When there is no delay in the induction of protective antibodies against a pathogen but the molecule inducing protective antibodies is known, it is possible to use a variation of the procedure described in Hypothetical example 1. In this case, a recombinant organism can be constructed where the protective molecule is absent or it is replaced by another molecule from another serotype of the same pathogen. In FIG. 2 it is shown an example of this method. Protein A could be replaced by protein A′ from a related pathogen. This recombinant pathogen is used as immunogen to inject an experimental animal. The antibodies that are obtained from this animal are used to negatively select a library of peptides. Concurrently, serum from an individual infected with the wild-type pathogen (which contains antibodies against protective epitopes in protein A) is positively selected against all epitopes present in the pathogen. This could be done using affinity purification. These antibodies (which are a mix of pathogen-specific protective and non-protective antibodies) are used to screen the library depleted of peptides binding to the non-neutralizing epitopes in all proteins (including A′) of the recombinant pathogen. As a result, only those phages carrying epitopes/mimotopes interacting with the antibodies against antigen A are selected. This procedure exemplified here with a phage library could be performed with different natural or synthetic libraries. Alternatively, instead of using a different protein (A′->A), areas of protein A could be modified in search of protective epitopes in the protein. A variation of this procedure would be the use of two closed-related pathogens that induce protective antibodies which do not cross-react.

In another preferred embodiment, the inventors have used the general method described above to determine a neutralizing epitope of PRRSV. After experimental infection with virulent PRRSV, viremia persists up to 40 dpi and antibodies appear at detectable levels around 9 dpi (Drew T W. Vet Res 31: 27-39, 2000). These antibodies target mainly ORF6 and ORF7, the matrix (M) and the nucleocapsid (N) protein respectively. However, neutralizing antibodies, which appear to be directed mainly to neutralizing epitopes present in glycoproteins GP5 are not detected until 35 or more dpi (Labarque, G. G., et al. J. Gen. Virol. 81:1327-1334,2000; Oleksiewicz, M. B.et al. Virology 15:135-140,2000). PRRSV's GP5 has two hydrophilic areas: one amino-terminal and one carboxy-terminal. Recently, it has been demonstrated that the carboxy-terminal region of GP5 of PRRSV does not carry neutralizing epitopes (Rodriguez M J, et al. J Gen Virol. 82:995-9, 2001).

We took advantage of the features of the antibody response against PRRSV's infection that produces a high level of non-neutralizing antibodies at the beginning of the infection and a delay in the induction of neutralizing antibodies. These non-neutralizing antibodies can be detected by ELISA. These antibodies were used to subtract a 12-mer random phage library. Then, a serum with a high level of neutralizing antibodies against PRRSV was used to positively select those phages carrying epitopes/mimotopes.

The invention will be further characterized by the following examples. These examples are not meant to limit the scope of the invention which has been fully set forth in the foregoing description.

### EXAMPLE 1

Affinity Purification of Anti-PRRSV Swine Antibodies and Monoclonal Antibody ISU25C1.

1. Affinity Purification of Anti-PRRSV Swine Antibodies

A non-neutralizing PRRSV-specific serum from early infection (less than 21 days) was used to subtract those phages expressing mimotopes resembling non-neutralizing epitopes. Neutralizing serum (NS) was prepared by hyperimmunization of sows with the following strains of PRRSV: PRRSV IA strain 97-7895 (1) (Genbank accession #:AF325691), PRRSV 16244B (Allende, R. et al, J. Gen. Virol. 80: 307-315,1999) (Genbank accesion #: AF046869), NVSL (obtained from NVSL, USDA, APHIS/Ames, Iowa), the American prototype PRRSV strain 2332 (American Type Culture Collection), modified-live vaccine virus RespPRRS (NOBL Labs.; Ames, Iowa) and modified-live vaccine virus Prime PacPRRS (Schering Plough Animal Health, Elkorn, Ne.).

A 1.5 ml volume of NS (s/p ratio by ELISA of 1.053; SN titer 1:64) was diluted 1:10 in 10 mM Tris pH 7.5 and passed three times through a MARC-145 (Kim, H S, Arch. Virol. 133(3-4): 477-483, 1993) cell column in which 30 mg of uninfected MARC-145 proteins had been coupled to CNBr-activated Sepharose (Sigma, St. Louis, Mo.). Non-binding antibodies (flow through) were recovered and passed three times through a cell-associated virus column in which 30 mg of MARC-145-PRRSV-infected cells had been coupled to CNBr-Sepharose. After extensive washing with 10 mM Tris pH 7.5 followed with 10 mM Tris pH 7.5/0.5M NaCl, specifically bound antibodies were eluted with 100 mM glycine pH 2.5 and pH was adjusted with 1 M Tris pH 8. A further purification step was performed by passing the antibodies through a protein A/G-Sepharose resin (Sigma, St. Louis, Mo.). After the purification, the solution enriched with anti-PRRSV neutralizing antibodies (NA), had a SN titer of 1:32. The absence of anti-MARC-145 cells antibodies in NA was confirmed by immunofluorescence.

2. Affinity Purification of Monoclonal Antibody ISU25C1

A 2-ml volume of ascitic fluid of hybridoma ISU25C1 (Yang, L et al, Arch. Virol. 145:1599-1619, 2000) was passed three times through a column bound to PRRSV-infected MARC-145 cells. Washing and elution steps were performed as said before for swine antibodies. Three fractions, containing a total amount of 68 μg of antibodies were dialyzed against PBS. The antibodies were then coupled to 50 mg of CNBr-activated Sepharose (Sigma, St. Louis, Mo.) following the manufacturer's indications.

These anti-PRRSV specific swine antibodies and anti-PRRSV specific monoclonal antibodies were used to select peptides expressed on the coat of bacteriophages. They were also used to characterize the selected phage clones and synthetic deduced peptides carried on their coats and variants of them.

### EXAMPLE 2

Biopanning of a 12-mer Library of Bacteriophages.

1. Biopanning Using Anti-PRRSV Specific Swine Antibodies

Three rounds of biopanning in solution were carried out. Briefly, 10 μl of the phage library (Ph.D12, NEB, Watertown, Mass.), were incubated for 30 minutes at room temperature with 1 μg of anti PRRSV affinity purified neutralizing antibodies. To capture the phage-antibody complexes, 50 μl of BSA-blocked protein A/G-Sepharose were added, and incubated for 15 minutes with antibody-phage complexes. The particles of protein A/G-Sepharose were then washed ten times with TBS containing increasing concentrations of Tween-20 in each panning (0.05, 0.1 and 0.5% in the first, second and third panning, respectively). Finally, phages were eluted through a 10-min incubation of the Sepharose in 100 mM glycine pH 2.5. Sepharose particles were centrifuged for 30 seconds, and the eluate was transferred to another tube. The pH was adjusted with Tris 1 M pH 8.5.

An aliquot of the eluted phages was titrated in E. coli ER2738, and the rest of the eluate was amplified for 4.5 hours in a mid log E. coli ER2738 culture. Phages were then precipitated with 3.33% PEG 8000 and 0.42 M NaCl for 16 hours at 4 C. and titrated.

The amplified enriched library was subjected to further cycles of selection. After the second panning was done with NA, a third cycle of selection was performed using non-neutralizing serum to substract those clones interacting with non-neutralizing antibodies. Those phages that did not bind to non-neutralizing serum, were selected once more with the neutralizing antibodies to further enrich for clones expressing peptides resembling neutralizing epitopes.

2. Biopanning Using Monoclonal ISU25C1 as Ligand

Two selections with different stringency conditions were performed with Monoclonal antibody ISU25-C1. In the first one, 2 μl of ISU25-C1 ascites fluid were diluted in 200 ul of 10 mM Tris pH 7.5 and incubated for 30 minutes with the Ph.D.12 phage library. Then, 50 μl of blocked Protein G-agarose were added to the phage-antibody mixture and incubated for 30 minutes with occasional mixing. The resin was washed with TBS containing 0.05% Tween-20 in the first panning, TBS containing 0.1% Tween-20 in the second panning and TBS containing 0.5% Tween-20 in third panning. Phages bound to ISU25C1 were eluted with 100 mM glycine pH 2.5.

For the second more stringent selection, 2 μl of ISU25C1 ascites fluid were diluted in 200 μl of Tris 10 mM pH 7.5 and incubated for 30 minutes with the Ph.D.12 phage library. Then, 50 μl of Protein G-agarose (50% aqueous solution) were added to the phage-antibody mixture and incubated for 30 minutes with occasional mixing. In the second panning, CNBr-activated-Sepharose-coupled Monoclonal antibody was used. In the third panning, protein G was used again to capture the phage-antibody complexes from solution.

The resin was thoroughly washed with Tris 10 mM pH 7.5 in the first panning, with 10 mM Tris pH 7.5 containing 0.5 M NaCl in the second panning, and with 10 mM Tris pH 7.5 containing 0.05% Tween-20 in the third panning.

3. Biopanning With Affinity Purifed Anti Peptide S2-antibodies

A peptide comprising residues 27-44 of the GP5 of PRRSV IA 97-7895 isolate was synthesized with the residues 27-30 being changed to the irrelevant motif SGSG (peptide S2, as shown in Table 1). Anti-peptide-S2 antibodies (peptide S2 is shown in Table 1) were affinity purified from anti-PRRSV pooled precipitated antibodies and 2 ug of them were used for each round of selection. Three pannings with protein A/G were performed. For the first one, PBS with 0.1% Tween-20 was used for the binding and washing steps. For the second and third pannings, PBS with 0.5% Tween-20 was used. Thus, a deduced peptide obtained from a bacteriophage library by selection with swine neutralizing antibodies and a neutralizing monoclonal antibody was used to further purify swine anti-PRRSV antibodies. These anti-S2 peptide purified antibodies were used to further enrich the phage library with phage clones carrying peptides interacting with the swine antibodies recognizing the area of GP 5 of PRRSV which elicits neutralizing antibodies.

TABLE 1

Sequences of the synthetic peptides
used throughout the experiments
described in this patent.

| | |
|---|---|
| Peptid | Sequence |

| | |
|---|---|
| S1^{e} | VLVNANNSSSSHFQSIYNC |
| S2 | SGSGANNSSSSHFQSIYNC |
| S3 | SGSGANNSSSSGSGSIYNC |
| S4 | SHITSYPAYFWC |
| S6 | SGSGANSNSSSHLQLIYNLTLCE |
| | LC |
| R4N | ALVNIPISNNLAC |
| R1N | VLVNSNNSSSSAC |
| Δ4R1 | SGSGSNNSSSSAC |
| N | |
| P7 | QRAYLELPPWPPC |

Peptides were synthesized using solid-phase technology and 9-fluorenylmethoxy carbonyl chemistry at the Protein Core Facility of the Center for Biotechnology, University of Nebraska.. All of them have a carboxyterminal cysteine which allows the coupling to MBS-activated proteins or ELISA plates.

4. Determination of the Deduced Sequence of the Peptide Carried on Selected Phages Clones

Non-amplified eluates were plated on LB top agar containing 50 μg/ml of IPTG and 40 μg/ml of Xgal, and after 16 hours of incubation, single phage plaques were amplified in 1 ml of mid log. E. coli ER2537 in LB medium for 4.5 hours at 37 C. After one precipitation with PEG 8000 as described, DNA from the phages was extracted and sequenced using -96 gIII sequencing primer (CCC TCA TAG TTA GCG TAA CG). Sequencing was performed manually using the dideoxy-mediated chain termination method by Sanger et al using T7 Sequenase version 2.0 DNA polymerase (Amersham Life Science, Inc) and [α-³⁵S]-dATP. Samples were run in gels containing 50% urea and 6% polyacrilamide. Gels were dried onto a 3MM paper and exposed to a film (Kodak-BioMax MR) during 36 hs at RT for autoradiographic detection of the sequence.

In FIG. 3 there are shown the sequences of selected clones recovered after the third panning of the first run of selection performed using monoclonal antibody ISU25-C1 (FIG. 3A). Three out of fifteen clones with a SH motif were found. In a second selection with Monoclonal antibody ISU25-C1 using more stringent conditions all phages in the third panning carried an identical peptide with a SHI motif (FIG. 3B). This SHI/L motif is found in residues 37-39 of GP5 of PRRSV isolate KY-35 which was the isolate used to immunize the mice for the production of Monoclonal antibody ISU25-C1.

Using anti-PRRSV affinity purified NA to select phages from the library, several clones carrying the aminoterminal motif ALVN were recovered (FIG. 3C). This motif is a consensus sequence found in residues 27-30 of GP5 of PRRSV (Dea, S Arch. Virol. 145:659-688, 2000). Another clone selected with neutralizing antibodies (clone 18), expressed a peptide with a YNL motif, which is also a consensus motif found in aminoacids 43-45 of GP5 of PRRSV. Finally, clone 14 carried a HF motif that is found in residues 39-40 of GP5 of PRRSV IA 97-7895 isolate which was the primary PRRSV strain used to immunize the pigs.

Anti-PRRSV precipitated antibodies with a SN titer of 1:512 were affinity purified with this peptide. The anti-peptide-S2 affinity purified antibodies were then used to select epitopes from the 12-mer-phage display library. Several copies of a clone with the sequence YKNTHLDLIYNA were selected (FIG. 3D). Motif HxxxIYN is found in all of the five PRRSV isolates used to immunize the pigs. Moreover, motif HLQLIYN is present between residues 38-44 of the PRRSV consensus sequence of ORF5 (Dea, S et al, Arch. Virol. 145:659-688,1998). In addition, motif HFQ, found in the peptide expressed by clone S2-12 is located at aminoacids 38-40 of PRRSV Iowa strain, indicating that some of the anti-peptide S2-affinity purified antibodies could also be recognizing the 2 aminoacids located downstream from H39.

Altogether, these results show that the neutralizing serum has antibodies directed to two putative epitopes located in the ectodomain of GP5. One of these epitopes (which we named epitope A) would comprise the stretch of aminoacids involving residues (²⁷A/V)LVN³⁰ of GP5. The other epitope (epitope B) would comprise the stretch of amino-acids involving residues H³⁹(F/L)⁴⁰XXI⁴³Y⁴⁴N⁴⁵. The SH(L/I) motif recognized by neutralizing Monoclonal antibody ISU25-C1 is located in the epitope B area.

### EXAMPLE 3

Recognition of Peptides Carried on Phage Clones by Anti-PRRSV Swine Sera and Monoclonal Antibodies.

To detect peptides on the coat of phages, individual clones shown in FIG. 3 were propagated for 4.5 hours in E. coli ER2738 and then they were purified by two precipitations with PEG 8000. 1010 phages were added to each well of a Immulon 2HB (Dynex Technologies Inc, Chantilly, Va.) in sodium bicarbonate 0.1 M pH 8.6, and incubated for 16 hours at room temperature in a humidified chamber. Plates were blocked with PBS containing 10% milk for three hours at room temperature. Swine sera or Monoclonal antibody from the tissue culture supernatant were preadsorbed in PBS containing 10% non-fat milk added with 100 μg/ml of E. coli proteins. In addition, 10¹⁰ pfu/ml of non-selected phages were added to the antibodies to reduce background caused by anti M-13 activity found in animal sera. The plates were washed six times with PBS 0.05% Tween-20 and the peroxidase-conjugated secondary antibody (1:500) was added for 30 minutes at 37 C. The plates were washed six times, and ABTS (KPL; Gaithersburg, Md.) substrate was added for 15 minutes. The results were read in a spectrophotometer at 405 nm. The specific reactivity of the serum against the phage-displayed peptide was calculated as the difference between the reactivity against the tested phage clone and the reactivity against phages carrying an irrelevant peptide.

Several phage clones were tested in a phage-ELISA format. FIG. 4 shows that the peptide displayed by phage clone 5.1, which carries a SH(I/L) motif corresponding to epitope B, was strongly recognized by Monoclonal antibody ISU25-C1. However, this peptide was not recognized by either swine anti-PRRSV NS or NNS. Conversely, phage clone 01 expressing a peptide with an ALVN amino-terminal motif corresponding to epitope A, was strongly recognized by anti-PRRSV neutralizing serum and non-neutralizing serum, but was not recognized by Monoclonal antibody ISU25-C1. In addition, the peptide displayed by phage clone S2-4, which carries a HxxxIYN motif and corresponds to epitope B, was strongly recognized by swine neutralizing serum but not by swine non-neutralizing serum or by monoclonal antibody ISU25-C1.

These results indicate that the neutralizing motif HxxxIYN is only recognized by a fraction of the neutralizing antibodies from swine. And, the monoclonal antibody cannot recognize the neutralizing epitope with which swine neutralizing antibodies interact. Although the recognition area for monoclonal antibody ISU25-C1 is located in the same area of the neutralizing epitope recognized by swine neutralizing antibodies, there are differences in the epitope recognize by this monoclonal antibody. Thus, the peptide carried on Phage Clone 5.1 (FIG. 3B) is a mimotope for the epitope which is recognized by Monoclonal antibody ISU25C1.

### EXAMPLE 4

Recognition of Synthetic Peptides by Anti-PRRSV Swine Sera and Monoclonal Antibodies.

Several deduced peptides and variations of them were synthesized using solid phase technology and Fmoc standart chemistry. To perform ELISAs with these synthetic peptides, they were diluted in PBS pH 6.8 to a final concentration of 50 μg/ml, and then were used to coat Reacti-Bind Maleimide-Activated Plates (Pierce, Rockford, Ill.) for 5 hours at room temperature. Remaining maleimide groups were blocked by a 1-hour incubation with cysteine-HCl 10 μg/ml diluted in PBS pH 6.8. An extra blocking step was carried out by incubating the plate with PBS containing 10% milk. Sera or affinity-purified antibodies were added in different dilutions and incubated for 1 hour at room temperature.

To perform competition assays, several dilutions of peptide S4 and irrelevant peptide P1 (shown in Table 1) were incubated with Monoclonal antibody ISU25-C1 for 1.5 hours at room temperature. Then, the antibody-peptide mix was added to PRRSV-coated ELISA strips (IDDEX Lab., Inc; Westbrook, Me.) for 45 minutes.

Monoclonal antibody ISU25-C1 recognized peptide S4, which was deduced from the inserted sequence of phage clone 5.1. The specificity of Monoclonal antibody ISU25-C1 for peptide S4 was assessed by an experiment of inhibition of binding in ELISA. In FIG. 5 it is shown that at a concentration of 333 μM, peptide S4 inhibited 60% of the binding Monoclonal antibody ISU25-C1 to PRRSV.

NS obtained from different immunized swine recognized peptides R4N and R1N. While peptide R4N corresponds to the sequence of the peptide expressed by phage clone 01, peptide R1N corresponds to aminoacids 27-38 of the GP5 of PRRSV IA 97-7895 isolate. Both peptides share a (A/V)LVN motif, which corresponds to aminoacids 27-30 of PRRSV's GP5. The signal was abrogated when these residues (27-30; VLVN in R1N peptide) were changed to an irrelevant motif SGSG in peptide ΔR1N as shown in FIG. 6.

Peptide S1 that corresponds to residues 27 to 43 of GP5 of IA 97-7895 and contains both motifs, VLVN and HXXXIYN, was strongly recognized by neutralizing serum from these pigs (as shown in FIG. 6). Peptide S2, in which motif VLVN was replaced with a SGSG motif, was still recognized by swine neutralizing serum, although with a considerable reduction in the ELISA signal. However, the signal was completely abrogated when motifs VLVN and SHF were replaced by motifs SGSG and GSS respectively (peptide S3), indicating that both of them are a key component of their respective epitopes.

### EXAMPLE 5

Anti-peptides-affinity Purified Antibodies Recognized Synthetic Peptides in ELISA.

Based on the previous results, anti-epitope A and anti-epitope B antibodies were affinity purified from a pool of precipitated antibodies with a SN titer of 1:512 using Keyhole-limpet hemocianine (KLH)-conjugated peptides coupled to CNBr-activated Sepharose as ligand as it is known in the art. Eluted antibodies were used to detect synthetic peptides in ELISA as shown in FIG. 7. R4N/R1N-affinity purified antibodies recognized peptides R4N, peptide R1N and peptide S1. All these peptides contain an (A/V)LVN motif. However, these antibodies did not recognize peptides S2 nor ARLN in which the N-terminal residues VLVN were changed to SGSG. Conversely, S2 affinity purified antibodies recognized peptides S1 and S2 containing the SHXXXIYN motif, but did not recognize peptides R4N, R1N nor S3, which lack this motif. Moreover, peptide S3 only differs from peptide S2 in that in the former, the motif SHF was replaced by GSG, suggesting a key importance of one of these three amino-acids for antibody binding.

The signal obtained in ELISA when using peptide S1 as antigen and a mix of affinity purified anti R4N/R1N and anti S2 antibodies together as primary antibodies, is higher than the signals obtained when using these antibodies separately (FIG. 7). Taken together, these results confirm the existence of two separate epitopes present in the GP5 ectodomain of PRRSV.

### EXAMPLE 6

Further Characterization of Epitope B.

The alignment of the deduced sequences of the peptides expressed by phages selected with monoclonal antibody ISU25-C1, swine neutralizing antibodies and anti peptide S2-affinity purified swine antibodies (FIG. 3) suggests that epitope B is composed of two stretches of aminoacids located between positions 37-39 (SHL/F) and between positions 42-45 (IYNL) of PRRSV GP5. To further characterize epitope B, inhibition experiments were performed with peptides S2 and S3 as shown in FIG. 8. Although both peptides inhibited the binding of anti-peptide S2-affinity purified antibodies to clone S2-4, peptide S2 inhibited to a greater extent than peptide S3. This stresses the importance of motif SHF in the composition of epitope B. Anti-peptide S2 affinity-purified antibodies, recognized to a similar extent peptides S2 (OD: 0.336) and S6 (OD: 0.369), confirming that residues H³⁸, Q⁴⁰, I⁴², Y⁴³ and N⁴⁴ constitute the core of epitope B.

### EXAMPLE 7

Difference in the Kinetics of Appearance of Antibodies Induced by Epitopes A and B.

Anti-PRRSV neutralizing antibodies appear late after infection and they are mainly directed against GP5. To determine a correlation between time post-infection and epitope recognition, we evaluated sera from several pigs at two different times of the hyperimmunization protocol as shown in FIG. 9.

Sera obtained shortly after the beginning of the immunization, which were positive to PRRSV by ELISA but negative or with low SN titer, reacted very strongly with epitope A (phage clone 01) and very weakly with epitope B (phage clone S2-4). Sera from the same animals were tested after repeated immunizations, when SN titers had reached 1:32 to 1:128. At these times, the response against epitope B had considerably increased. To the contrary, the signal to epitope A remained constant or decreased after several immunizations. Thus, epitope B is a neutralizing epitope which do not induce antibodies until three or more weeks after infection.

Aminoacids ²⁷A/(V)LVN³⁰ of GP5 are the main recognition site for epitope A. The substitution of these four aminoacids with an irrelevant motif (SGSG) abrogates its recognition (FIGS. 6 and 7). This epitope A was strongly recognized by swine sera obtained early after infection, when non-neutralizing activity was detected. Later, as the immune response progressed, the titer of antibodies against this epitope decreased in most of the sera from hyperimmunized pigs tested in these experiments (FIG. 9). Nevertheless, even when high SN titers are detected in sera, epitope A elicits more antibodies than epitope B. This observation explains why, despite having performed a subtraction of non-neutralizing epitopes from the 12-mer phage display library with non-neutralizing serum, several clones displaying mimotopes of epitope A were selected with neutralizing serum (FIG. 3).

The region of GP5 where epitope A is located was shown to be hypervariable. This region also has a high percentage of accessible residues, as determined by Janin algorithm (Janin, J, Nature 277:491-492.1979). It has been proposed that the presence of hypervariable regions within the hydrophilic domains of GP5 can be the result of the host's selective humoral immune response against this region, favoring antigenic drift (Pizardeh, B and S Dea, J. Gen. Virol. 79:989-999, 1998). However, as determined here, epitope A is not associated with viral neutralization. Nevertheless, the early and intense antibody-response elicited by epitope A could be associated with a viral evasion mechanism. Epitope A acts as a decoy epitope, luring the immune response away from more protective targets in GP5. Indeed, it exhibits some features expected of a decoy epitope, such as its high immunogenicity expressed rapidly after infection, hypervariability, and absence of correlation between seroconversion and appearance of NA (Garrity R R et al, J. Immunol. 159:279-289,1997). The site of cleavage of the GP5 signal peptide is not known. Using different prediction methods, some researchers postulated that the site of cleavage is located between aminoacids 31-32 (Andreyev V G, Arch. Virol. 142:993-1001,1997), while others suggested that it is located between aminoacids 25-26 (Dea S et al, Arch. Virol. 142:993-1001, 2000). The constant location of the ALVN at the N-terminus in the four different phagotopes selected with swine serum shows that a form of GP5 with this motif at the aminoterminus is present during infection with PRRSV.

Epitope B is located in a region of GP5 that comprises residues 38-45. This region is recognized both by neutralizing Monoclonal antibody ISU25-C1 and swine sera with high neutralizing titers but not by swine non-neutralizing sera. Our finding of a sequential epitope agrees with previous reports indicating the ability of Monoclonal antibody ISU25-C1 to recognize the denatured GP5 (Yang Let al, Arch. Virol. 145:1599-1619, 2000). Furthermore, sequential neutralizing epitopes in GP5 of PRRSV have also been described by others (Pizardeh, B and S Dea, J. Gen. Virol. 78:1867-1873, 1997). Sequence comparison of several PRRSV isolates demonstrates that the area where epitope B is located is conserved. Moreover, anti-peptide S2-affinity purified antibodies recognized to a similar extent the epitope variants present in peptides S2 (HFQSIYN) and S6 (HLQLIYN) and the mimotope carried by phage clone S2-4. This could explain the broad neutralizing activity of Monoclonal antibody ISU 25-C1 (Yang Let al, Arch. Virol. 145:1599-1619, 2000). In addition, these results clearly demonstrate that residues H³⁹ and I⁴³Y⁴⁴N⁴⁵ are the main recognition site in epitope B. Nevertheless, residues 40-42 could also be involved in binding to antibodies, since the peptide carried on phage clone S2-4 contains leucines 40 and 42.

Residue H³⁹ in PRRSV GP5 is also relevant for binding of swine antibodies to epitope B, since the substitution of H?G completely abrogated the-recognition (FIGS. 6 and 7). The use of anti-peptide S2-affinity purified antibodies confirmed this assertion, since using these antibodies to select epitopes from the phage library, phage clone S2-4 was highly enriched. This phage clone carries a peptide with a HLDLIYN motif. (FIG. 3). The high degree of conservation among PRRSV isolates in the area where epitope B is located, suggests that this region has a important functional or structural role for these viruses. Thus, the number of viable mutations in this area may be limited.

The titer of anti-epitope B antibodies in sera is low (FIG. 9) at time point A at the beginning of the hyperimmunization protocol, when non-neutralizing antibodies are undetectable (<1:4) but S/P ratio in ELISA (IDEXX Laboratories Inc; Westbrook, Me.) was at least higher than 0.652. Later, at time point B, when seroneutralization titer determined by fluorescence foci neutralization assay increased to >1:32, antibodies against epitope B dramatically rise.

Despite being separated by only 7 aminoacids, both antigenic regions identified here are independent epitopes. In FIG. 7 it is shown that both epitopes can be recognized simultaneously by antibodies, without any interference between them. Moreover, the increase in the titer of antibodies against epitope B correlates with a decrease in the level of antibodies against epitope A (FIG. 9).

### EXAMPLE 8

Lactate-Dehydrogenase Virus has a Neutralizing Epitope in the Same Area as PRRSV

Epitope B of PRRSV is located in a region of GP5 that comprises residues 37-45. This region is recognized both by neutralizing Monoclonal antibody ISU25-C1 and swine sera with high neutralizing titers but not by swine non-neutralizing sera. Sequence comparison of several PRRSV isolates demonstrates that the area where epitope B is located is conserved. Moreover, anti-peptide S2-affinity purified antibodies recognized to a similar extent the epitope variants present in PRRSV IA 97-7895(HFQSIYN) and PRRSV KY-35 (HLQLIYN), two highly divergent North American isolates. In addition, these antibodies recognized the mimotope carried by phage clone S2-4 (HLDLIYN). These observations explain the broad neutralizing activity of Monoclonal antibody ISU 25-C1 (Yang, L. 2000. Arch. Virol. 145:1599-1619. Additionally, these results demonstrate that residues H³⁸ and I⁴³Y⁴⁴N⁴⁵ are the main recognition site in epitope B. Nevertheless, it seems that residues 39-42 contribute to the binding to antibodies, since the peptide carried on phage clone S2-4 contains leucines 40 and 42, which are also present in PRRSV isolate KY-35. This finding regarding the presence of a sequential neutralizing epitope is in agreement with previous reports indicating the ability of Monoclonal antibody ISU25-C1 to recognize the denatured GP5 (Yang, L., et al. Arch. Virol. 145:1599-1619, 2000). Furthermore, the presence of sequential neutralizing epitopes in GP5 of PRRSV has also been described by others (Pirzadeh, B. and S. Dea. J. Gen. Virol. 78:1867-1873,1997).

The product of ORF5 of lactate dehydrogenase-elevating virus (LDV), a murine arterivirus, has more than 60% similarity at the aminoacid level with the GP5 of PRRSV as it is shown in FIG. 10. A neutralizing epitope located between residues 37-60 of LDV ORF5 product was described (Li, K., et al. Virology 242:239-245, 1998). The key residues in this epitope were identified. Interestingly, they correspond to the residues identified by us as part of the main recognition site for NA in epitope B. For example, threonine³⁹ of the ORF5 product of LDV, which corresponds to histidine³⁸ in PRRSV GP5, seems to be of key importance in the recognition by anti-LDV neutralizing monoclonal antibodies 159-12 and 159-18 as it was the only residue mutated (threonine->_alanine) in a LDV neutralization escape mutant. Moreover, neutralizing monoclonal antibody 159-19 (which reacts with the same epitope recognized by monoclonal antibodys 159-12 and 159-18) recognized peptides with substitutions in I⁴³ or Y⁴⁴ to a lesser extent than the original peptide, indicating that both residues contribute to this epitope. Fine characterization of the neutralizing epitope of LDV indicated that it comprises residues 30 to 50 of the envelope glycoprotein (Plagemann, PGW, Virology 290 11-20,2001). It seems that residue H³⁸ in PRRSV GP5 is also relevant for binding of swine antibodies to epitope B, since the substitution of histidine by glycine completely abrogated the recognition (FIGS. 6, 7 & 8). The use of anti-peptide S2-affinity purified antibodies confirmed this assertion, since the use of these antibodies to select epitopes from the phage library resulted in a significant enrichment of phage clone S2-4. This phage clone carries a peptide with a HLDLIYN motif (FIG. 3). Thus, epitope B seems to be located in the same area of GP5 of the LDV neutralizing epitope.

### EXAMPLE 9

Conservation of Neutralizing Epitopes B among Isolates of PRRSV.

Only a fraction of the repertoire of B cell epitopes presented by a protein antigen is functionally capable of eliciting antibodies. It has been proposed that the chemical composition of a given epitope could influence at least one of the parameters that defines its immunodominance (Nakra, P., et al. J Immunol. 164:5615-5625, 2000). In fact, analysis of the deduced amino-acid sequence between residues 37 to 45 of GP5 (which are the main recognition region for epitope B) of 166 PRRSV isolates in North America (Table 2) and 106 isolates in Europe (Table 3) reported in Genbank to date, shows a high degree of conservancy in epitope B. The most common sequence includes a Leucine (L) in position 39, but the Iowa isolate 97-7985 (a phylogenetically distant isolate from 16244B) has a Phenylalanine (17% of GP5 sequenced isolates). There are also isolates with changes in Histidine in position 38 by Glutamine (2.9%), Asparagine (2.9%), Lysine (1%) and Tyrosine (0.5%).

TABLE 2

| | |
|---|---|
| Sequence | Quantity |

| | |
|---|---|
| SHLQLIYNL | 110 |
| SHFQLIYNL | 24 |
| SHLQLIYIL | 7 |
| SNLQLIYNL | 7 |
| SHFQLSYNL | 5 |
| SHSQLIYNL | 4 |
| SHVQLIYNL | 3 |
| SKLQLIYNL | 2 |
| SHLQLINNL | 2 |
| SQLQLIYNL | 1 |
| SNFQLIYNL | 1 |
| Total | 166 |

TABLE 3

| | |
|---|---|
| Sequence | Quantity % |

| | |
|---|---|
| STYQYLIYNL | 102 |
| SQLQSLIYNL | 2 |
| SYSQLLIYNL | 2 |
| Total | 106 |

On the other hand aminoacids 27-30 of GP5, which constitute the main recognition region of decoy epitope A present several variants: VLVN (45%), VLAN (22%), ALVN (7%), VLVS (15%), VIVN (8%) and VLVG, VPAN, ALAN, VLPN, VLAD, ALVS, VLVD (between 1 and 3% each) in American isolates. Thus, it looks that epitope A had evolved to behave as a decoy epitope, luring the immune response away from more protective targets present in GP5 such as neutralizing epitope B. Indeed, epitope A exhibits some features expected of a decoy epitope (Garrity, R. R. et al. J. Immunol. 159:279-289, 1997), such as its high immunogenicity expressed rapidly after infection and its hypervariability. In other viral infections, decoy epitopes have been described to act by suppressing the recognition of neutralizing epitopes.

For example, dampening of an immunodominant epitope on gp120 of HIV, allowed the refocusing of the neutralizing antibody response towards a previously silent, qualitatively different, second-order neutralizing epitope (Garrity, R. R. et al. J. Immunol. 159:279-289,1997). In addition, neutralizing epitope ERDRD on gp41 of HIV-1 was demonstrated to be immunogenic in rabbits. However, this response is completely abrogated when epitope ERDRD is presented on a peptide juxtaposed to the immunodominant epitope IEEE, located at a two-aminoacids distance in the native sequence of gp41 (Cleveland, S. M, et al. Virology. 266:66-78, 2000). Thus, the decoy epitope IEEE decreases the antibody response to the ERDRD neutralizing epitope. Epitope A of PRRSV, which is located 7 aminoacids apart from the neutralizing epitope B, could be acting in a similar fashion to the ERDRD epitope of HIV.

The lower variability observed in epitope B is consistent with the strategy that we postulate PRRSV follows early in infection. During the first weeks post-infection the antibody response against the ectodomain of GP5 is mostly centered in the immunodominant and non-neutralizing epitope A. This provides a window of time throughout which the PRRSV replicates profusely in the host, sheds and infects other contact naive animals. At a later time in this process, the induction of neutralizing antibodies together with the cellular immune response eventually eliminate the virus from the organism (Allende R, et al. J Virol. 74(22):10834-7,2000). At this time mutations in epitope B would not represent an evolutionary advantage to the virus.

It is possible that some of the antibodies anti-epitope B are directed against the sugar added to N. Epitope B, is located in an region of the GP5 molecule in association with a glycosylation site which is very conserved among isolates. Previous observations indicated that Monoclonal antibody ISU 25-C1 does not recognize GP5 expressed in E. coli (R. Bastos, personal communication). This site is sensitive to endoglycosidase F (Mardassi H, et al. Virology. 221(1):98-1 12, 1996) indicating the presence of N-linked oligosacharides and is also sensitive to some extent to endoglycosidase H which cleaves high-mannose saccharides.

To check if the inability of Monoclonal antibody ISU25-C1 to recognize its epitope was due to a lack of proper folding in the protein expressed in E. coli or to the need for glycosylation in the epitope instead, we digested PRRSV proteins with endoglycosidase F as shown in FIG. 11.

After digestion with endoglycosidase-F, the degree of recognition of Monoclonal antibody ISU25-C1 to PRRSV's GP5 in a Western Blot decreases dramatically indicating that the glycosylation in epitope B is involved to some extent in recognition by the mouse antibody. Thus, peptide S4 (Table 1) is a true mimotope which resembles some of the aminoacids SHL/F and, to some extent, the sugar present in the adjacent glycosylation site (N⁴⁵). This finding explain why ISU25 does not recognize peptide S2 (epitope A removed, epitope B present) and vice versa, pig sera with neutralizing antibodies do not recognize S4 (as shown in FIG. 6). ISU25-C1 uses an antibody VH gene that is the most phylogenetically distant to the only family of VH antibodies used by pigs (Butler J E, et al. Immunology 100: 119-30, 2000). This observation stresses the need of using antibodies from the same species target for the infection to determine the neutralizing epitope important for protection against a pathogen. Thus, although they may be some swine antibodies recognizing the sugar in epitope B, others recognize mainly the main amino-acids involved in this epitope.

### EXAMPLE 10

Positive Correlation Between Anti-epitope B Antibodies and Neutralizing Antibodies.

Commercial ELISA kits allow the detection of the presence of anti-PRRSV antibodies in serum. For instance, IDDEX ELISA kit (Iddex Laboratories Inc., Westbrook, Me.) has an OD (S/P) cut-off of 0.4 and it allows the determination of the status of immunization or infection of the herd. However, this ELISA does not distinguish between infected and vaccinated pigs. In addition, it does not allow the determination of the amount of antibodies that are neutralizing (protective) in the serum.

Using sera from experimentally infected pigs these Inventors demonstrated that there is a correlation between the rise of SN antibodies and the titer by ELISA using ELISA plates sensitized with peptide S2 (epitope A deleted, epitope B present) (FIG. 9).

During the first weeks post-infection the antibody response against the ectodomain of GP5 is mostly centered in the immunodominant and non-neutralizing epitope A. This provides a window of time throughout which the PRRSV replicates profusely in the host, sheds and infects other contact naive animals. At a later time in this process, the induction of neutralizing antibodies together with the cellular immune response eventually eliminate the virus from the organism (Allende R, et al. J Virol74(22):10834-7, 2000). At this time mutations in epitope B would not represent an evolutionary advantage to the virus.

A rapid method to determine the levels of neutralizing antibodies in a herd will have significant clinical value. However, testing the titer of neutralizing antibodies in a herd using classical virology techniques is costly and time-consuming.

Our objective was to evaluate whether there is a correlation between titer by SN and peptide ELISA using epitope B (peptide S2) with sera from the field as it was found in experimentally infected pigs (FIG. 9). We determined the seroneutralizing titers using the fluorescence foci neutralization assay (FFNA) assay. It was performed as described (Wu, W-H, Y. et al. Virology, 287(1):183-191, 2001). Serial dilutions of test sera were incubated for 60 minutes at 37° C. in the presence of 100 foci forming units of the virus in DMEM, containing 5% FCS. The mixtures were added to 96-well microtitration plates containing confluent MARC-145 cells which had been seeded 72 h earlier. After incubation for 24 h at 37° C. in a humidified atmosphere containing 5% CO₂, the cells were fixed for 5 minutes with a solution of 50% methanol, 50% acetone. After extensive washing with PBS, the expression of N protein of PRRSV was detected with Monoclonal antibody SDOW17 (Nelson, E. A at al, 1993, J. Clin. Microbiol. 31: 3184-3189,1993) followed by incubation with FITC-conjugated goat anti-mouse IgG (Sigma, St Louis, Mo.). Neutralization titers were expressed as the reciprocal of the highest dilution that inhibited 90% of the foci present in the control wells.

Irrelevant peptide P7 was used as a negative control for subtraction of the signal obtained with peptides S2 and R1N.

There is no correlation between the titers obtained by ELISA/peptide R1N (epitope A) and those obtained by SN (p<0.1). Moreover some of the sera do not recognize peptide R1N. These results are expected for a highly variable decoy as is epitope A. However, we cannot confirm this assumption because no viral isolate was recovered from these cases. Nevertheless, there is an increase in titer with peptide S2 (epitope A deleted, epitope B present) when the SN titer is above 4. There is also a significant statistical difference between the populations with different SN titers and the value of their titers by S2-ELISA (p<0.1).

These results show that sera from different samples from the field recognize peptide S2, confirming the high degree of conservation of epitope B. It is possible that some of the viruses carry the mutations described for epitope B. Nevertheless, these sera are still able to recognize the epitope. Thus, it is possible to semi-quantify the amount of SN antibodies in a serum using peptide S2 ELISA and positively correlate this with protection.

In summary, at least two epitopes exist in the ectodomain of GP5 of PRRSV. Epitope A is immunodominant and induces a rapid rise of antibodies without neutralizing activity. Thus, epitope A is a decoy epitope which hampers the immune response to PRRSV. Neutralizing antibodies targeting epitope B appear later in the immune response and in are in low amounts, when PRRSV is being cleared from the animal. Epitope B is conserved among isolates making it a suitable target for a more efficient vaccine for PRRSV and for the determination of the status of protection of an animal and the quality of a vaccine against PRRSV.

Thus, a vaccine formulation such as a peptide vaccine, a partial protein vaccine, a DNA vaccine (supplemented or not with immunogenic compounds such as cytokines etc), or an infectious vaccine or other formulation capable of presenting the epitopes in an immunogenic manner, should carry the epitope B and not the epitope A. An example of the peptides of neutralizing epitopes that could be used for the uses described above are mimotope SHITSYHPAYFW, and peptides SH(hydrophobic)D/Q(hydrophobic)(hydrophobic)YNL, or a peptide carrying the core of this peptide such as H(hydrophobic)D/Q(hydrophobic)(hydrophobic)YN for the American Isolates and STYQLIYNL for the European isolates.

### EXAMPLE 11

Use of Epitope B for Quality Control of Vaccines.

In the process of making a vaccine it is of the most importance to determine the presence of those epitopes inducing the protective immunity in the final product and at different stages of the production. The knowledge of the importance of this epitope B in induction of protection will be of great value for quality control of vaccines. Thus, antibodies can be prepared against this epitope and used to determine that epitope B is not lost during the different steps in the production of a vaccine. The degree of reaction in a test such as an ELISA where the antigen for the vaccine is probed with these anti-epitope B antibodies will help the manufacturer to determine the quality of the vaccine that it is being produced. An example of a peptide to be used for this quality control test will be SH(hydrophobic)D/Q(hydrophobic)(hydrophobic)YNL.

### EXAMPLE 12

Use of Anti-epitope B Neutralizing Antibody to Induce Instant Protection in Pigs.

Vaccination takes time until the immune system of the animal mounts a response to the virus. In some cases it is necessary to give “instant” immunity to a herd for example in the case of an outbreak with PRRSV. In this case, the use of an anti-epitope B mono-specific antibody could help in hampering the infection with the virus. In addition, at the same time the antibody is administered, a vaccine carrying a different neutralizing epitope can be used to actively immunized the animal. This can be performed using mouse monoclonal antibodies or chimeric (mouse×swine) or swine antibodies obtained by recombinant DNA technology. There are several technologies dealing with the construction and expression of antibodies in different systems that can be applied to produce anti-epitope B antibodies with this purpose.

Although a preferred embodiment of the invention has been disclosed with some particularity, many modifications and variations in the invention are possible without deviating from the invention. Therefore, it is to be understood, that within the scope of the appended claims, the invention can be practiced other than as specifically described.

What is claimed is:

1. A method of isolating neutralizing epitopes of a pathogen, comprising the steps of
obtaining sera with and without neutralizing antibodies from a species; and
using the difference between the serum with neutralizing antibodies and the serum without neutralizing antibodies to isolate neutralizing epitopes or mimotopes or both epitopes and mimotopes.

2. A method according to claim 1 in which the step of using the difference between the serum with neutralizing antibodies and the serum without neutralizing antibodies to isolate neutralizing epitopes or mimothopes or both epitopes and mimotopes includes the step of removing the non-neutralizing antibodies against these epitopes or mimotopes or both epitopes and mimotopes from the serum having both neutralizing antibodies and non-neutralizing antibodies.

3. A method according to claim 1 in which the step of using the difference between the serum with neutralizing antibodies and the serum without neutralizing antibodies to isolate neutralizing epitopes or mimothopes or both epitopes and mimotopes includes the step of selectively removing the neutralizing antibodies against the epitopes or mimotopes or both epitopes and mimotopes from the serum having both neutralizing antibodies and non-neutralizing antibodies.

4. A method according to claim 1 in which the step of using the difference between the serum with neutralizing antibodies and the serum without neutralizing antibodies to isolate neutralizing epitopes or mimothopes or both epitopes and mimotopes includes the steps of:
using the serum without neutralizing antibodies to deplete a library of molecules which may contain epitopes or mimotopes or both epitopes and mimotopes that interact with non-neutralizing antibodies; and
using the subtracted library to positively select any epitopes or mimotopes or both epitopes and mimotopes from the subtracted library.

5. A method according to claim 4 in which the step of using the difference between the serum with neutralizing antibodies and the serum without neutralizing antibodies to isolate neutralizing epitopes or mimothopes or both epitopes and mimotopes further includes the steps of:
obtaining the sequence of the neutralizing epitopes or mimotopes or both epitopes and mimotopes; and
using the sequences of neutralizing epitopes or mimotopes or both epitopes and mimotopes to prepare neutralizing epitopes or mimotopes or both epitopes and mimotopes.

6. A method according to claim 1 in which the step of obtaining sera with and without neutralizing antibodies from a species includes the step of obtaining sera from infected animals.

7. A method according to claim 1 in which the step of obtaining sera with and without neutralizing antibodies from a species includes the step of obtaining sera from vaccinated animals.

8. A method according to claim 1 in which the step of obtaining sera with and without neutralizing antibodies from a species includes the step of obtaining sera after infection of the animal with a recombinant virus.

9. A method to determine neutralizing epitopes of a pathogen, comprising the steps of
obtaining sera with and without neutralizing antibodies from a species;
using the serum without neutralizing antibodies to deplete a library of molecules which may contain epitopes or mimotopes or both epitopes and mimotopes that interact with non-neutralizing antibodies;
using the serum with neutralizing activity to positively select any epitopes or mimotopes or both epitopes and mimotopes of the subtracted library;
obtaining the sequence of the epitopes or mimotopes or both epitopes and mimotopes; and
characterizing these sequences with serum with and without neutralizing activity.

10. A method to determine neutralizing epitopes of a pathogen, comprising the steps of
obtaining a monoclonal antibody recognizing a protective epitope of the pathogen; and
using said monoclonal antibody to select for epitopes or mimotopes or both epitopes and mimotopes from a display library.

11. A method to determine neutralizing epitopes of a pathogen, comprising the steps of
obtaining a monoclonal antibody recognizing a protective epitope of the pathogen; and
obtaining the sequence of these epitopes/mimotopes by sequencing and comparison to the known sequence of the proteins of the virus.

12. A method to determine neutralizing epitopes of a pathogen, comprising the steps of
obtaining a monoclonal antibody recognizing a protective epitope of the pathogen;
using the epitopes/mimotopes obtained in b) to affinity purify antibodies from immune serum of the species target for the infection with the virus; and
using these selected antibodies to screen a display library for peptides/mimotopes recognized by the antibodies of the host.

13. A method to determine neutralizing epitopes of a pathogen, comprising the steps of
obtaining a monoclonal antibody recognizing a protective epitope of the pathogen; and
using the epitopes/mimotopes obtained in b) to affinity purify antibodies from immune serum of the species target for the infection with the pathogen.

14. A method to determine protective epitopes of pathogens, comprising the steps of:
obtaining sera with and without protective antibodies from the species target for the pathogen or another species;.
using the serum without protective antibodies to deplete a library of molecules of epitopes or mimotopes or both epitopes and mimotopes interacting with non-protective antibodies;
using the serum with protective activity to positively select a epitopes or mimotopes or both epitopes and mimotopes of the subtracted library;
obtaining the sequence of these epitopes or mimotopes or both epitopes and mimotopes by sequencing and comparison to the known sequence of proteins of the pathogen; and
characterizing these sequences with serum with and without protective activity.

15. A vaccine comprising an effective dose including at least one neutralizing epitope against a pathogen without non-neutralizing epitopes.

16. A method for vaccination against Porcine Reproductive and Respiratory Virus using a subunit vaccine using PRRSV GP5 which will include the main component for epitope B one of S³⁷HLQLIYNL⁴⁵ and STYQYLIYN or their known equivalents and which does not include the main component or part of it of epitope A which is one of amino-acids 27-30 of GP5 of PRRSV and its known equivalents.

17. The vaccine of claim 16 wherein the immunogen is a polynucleotic vaccine which will not include the main component or part of it of epitope A.

18. The vaccine of claim 16 wherein the immunogen could be a recombinant virus expressing PRRSV GP5 which does not include the main component or part of it of epitope A.

19. A polypeptide vaccine including the main component for epitope B which includes amino acids 37 to 45 of GP5 and is free of the main component or part of it of epitope A which includes amino-acids 27-30.

20. A diagnostic ELISA or equivalent test including the main component of epitope B which includes at least one of aminoacids 37 to 45 of GP5 to allow determination of antibodies in serum against this particular epitope.

21. A differential diagnostic test including the main component of epitope B which includes at least one of aminoacids 37 to 45 of GP5 and another antigen which elicits antibodies soon after infection.

22. An ELISA or equivalent test including the main component of epitope B which includes at least one-of amino acids 37 to 45 of GP5 to allow determination of the quality of an immunogen present in a vaccine.

23. An ELISA or equivalent test including the main component of epitope B which includes at least one of amino acids 37 to 45 of GP5 to allow the determination of the induction of antibodies in an animal for the purpose of a potency test for a vaccine.

24. The use of polyclonal or monoclonal antibodies specific to epitope B to facilitate cure of infected animals or prevention of animals at risk of infection with PRRSV.

25. The use of monospecific chimeric 9 mouse×porcine antibodies obtained by recombinant DNA technology specific to epitope B to facilitate cure of infected animals or prevention of animals at risk of infection with PRRSV.

26. The use monospecific porcine antibodies obtained by recombinant DNA technology specific to epitope B to facilitate cure of infected animals or prevention of animals at risk of infection with PRRSV.
